SEQUENCE LISTING (10 sequences)

SEQ ID NO: 1 - DNA, 426 bp, Homo sapiens
gactcacagcccacagagttccacctgctcacaggttggctggctcagccaaggtggtgccctgctctgagcattcaggccaagcccatcctgcaccatggccaggtacagatgctgtcgcagccagagccggagcagatattaccgccagagacaaagaagtcgcagacgaaggaggcggagctgccagacacggaggagagccatgaggtgctgccgccccaggtacagaccgcgatgtagaagacactaattgcacaaaatagcacatccaccaaactcctgcctgagaatgttaccagacttcaagatcctcttgccacatcttgaaaatgccaccatccaataaaaatcaggagcctgctaaggaacaatgccgcctgtcaataaatgttgaaaagtcatcccaaaaaaaaaaaaaaaaaa

SEQ ID NO: 2 - protein, 51 aa, Homo sapiens
MARYRCCRSQSRSRYYRQRQRSRRRRRRSCQTRRRAMRCCRPRYRPRCRRH

SEQ ID NO: 3 - DNA, 683 bp, Homo sapiens
agaccagaccaacagtaacaccaagggcaggtgggcaggcctccgccctcctcccctactccagggcccactgcagcctcagcccaggagccaccagatctcccaacaccatggtccgataccgcgtgaggagcctgagcgaacgctcgcacgaggtgtacaggcagcagttgcatgggcaagagcaaggacaccacggccaagaggagcaagggctgagcccggagcacgtcgaggtctacgagaggacccatggccagtctcactataggcgcagacactgctctcgaaggaggctgcaccggatccacaggcggcagcatcgctcctgcagaaggcgcaaaagacgctcctgcaggcaccggaggaggcatcgcagaggctgcagaaccaggaagagaacatgcagaaggcactaagcttcctgggcccctcacccccagctggaaattaagaaaaagtcgcccgaaacaccaagtgaggccatagcaattcccctacatcaaatgctcaagcccccagctggaagttaagagaaagtcacctgcccaagaaacaccgagtgaggccatagcaactcccctacatcaaatgctcaagccctgagttgccgccgagaagcccacaagatctgagtgaaatgagcaaaagtcacctgcccaataaagcttgacaagacactc

SEQ ID NO: 4 - protein, 102 aa, Homo sapiens
MVRYRVRSLSERSHEVYRQQLHGQEQGHHGQEEQGLSPEHVEVYERTHGQSHYRRRHCSRRRLHRIHRRQHRSCRRRKRRSCRHRRRHRRGCRTRKRTCRRH

SEQ ID NO: 5 - DNA, 415 bp, Homo sapiens
gcccctcattttggcagaacttaccatgtcgaccagccgcaaattaaagagtcatggcatgaggaggagcaagagccgatctcctcacaagggagtcaagagaggtggcagcaaaagaaaataccgtaagggcaacctgaaaagtaggaaacggggcgatgacgccaatcgcaattaccgctcccacttgtgagcccccagcgggctctgccctggtgcgcttcacacagcaccaagcagcaacaagaacagcagaaggggaactgccaaggagacctgatgttagatcaaagccagagaggagcctatggaatgtggatcaaatgccagttgtgacgaaatgaggaatgtatatgttggctgtttttccccaacatctcaataaaactttgaaagcagaaaaaaaaaaaaaaaa

SEQ ID NO: 6 - protein, 55 aa, Homo sapiens
MSTSRKLKSHGMRRSKSRSPHKGVKRGGSKRKYRKGNLKSRKRGDDANRNYRSHL

SEQ ID NO: 7 - DNA, 599 bp, Homo sapiens
aggatgaggaggaggccctgcccctccaaacgtggcctcctatggacacccagactcacagccttcctatcacccacactcagctccatagcaactctcagccccaaagccgcacctgcacccgccattgccaaaccttcagccagagttgcagacagagccatcgtggcagccggagccagagctccagccagagcccggccagccaccgcaacccaactggagcccacagctcatccggccaccagagccagagtcccaacactagtccaccaccaaagcgccacaaaaagactatgaactcccaccactctcccatgcggcccaccatcctgcactgccgctgccccaagaacagaaagaacttggaaggcaagctgaaaaagaaaaaaatggccaagaggatccagcaggtgtacaaaaccaagacgcggagctcaggatggaaatccaactaatgagaccgcactccttggcttgttcctgcgtgtttcacccaaaggagaaaatgctaggatgaagtcaatcttcttgcaggaacatgttactatggtgatttctacgcaacactaattaaagcttgtacctggaagactaaa

SEQ ID NO: 8 - protein, 138 aa, Homo sapiens
MDTQTHSLPITHTQLHSNSQPQSRTCTRHCQTFSQSCRQSHRGSRSQSSSQSPASHRNPTGAHSSSGHQSQSPNTSPPPKRHKKTMNSHHSPMRPTILHCRCPKNRKNLEGKLKKKKMAKRIQQVYKTKTRSSGWKSN

SEQ ID NO: 9 - DNA, 19 bp, Artificial Sequence
  Synthetic Primer
atggccagataccgatgct

SEQ ID NO: 10 - DNA, 18 bp, Artificial Sequence
  Synthetic Primer
cagcatcttcgcctcctc

(19) United States
(12) Patent Application Publication
(10) Pub. No.: US 2017/0204433 A1
(43) Pub. Date: Jul. 20, 2017

(54) METHOD FOR RECONSTRUCTING A NON-HUMAN ANIMAL EMBRYO

(71) Applicant: UNIVERSITÀ DEGLI STUDI DI TERAMO, Teramo (TE) (IT)

(72) Inventors: Pasqualino LOI, Nepezzano (TE) (IT); Domenico IUSO, Foggia (FG) (IT); Marta CZERNIK, Alba Adriatica (TE) (IT); Federica ZACCHINI, Teramo (TE) (IT); Grazyna PTAK, Nepezzano (TE) (IT); Saadi KHOCHBIN, La Tronche, Grenoble (FR); Antonella FIDANZA, Teramo (TE) (IT)

(21) Appl. No.: 15/304,964

(22) PCT Filed: Apr. 22, 2015
(86) PCT No.: PCT/EP2015/058701

§ 371 Date: Oct. 18, 2016

Related U.S. Application Data
(60) Provisional application No. 61/982,357, filed on Apr. 22, 2014.

(51) Int. Cl. C12N 15/873; A01K 67/027; C12N 15/877

(52) U.S. Cl. CPC C12N 15/873; C12N 15/8773; A01K 67/0273; A01K 2217/00; A01K 2227/103

(57) ABSTRACT

The present invention refers to a method for reconstructing a non-human animal embryo, to a method for generating a nonhuman animal and to a non-human animal or non-human animal reconstructed embryo obtainable by the methods according to the invention. The method of somatic cell nuclear transfer (SCNT) has a step of transiently expressing or inducing the uptake of a protein involved in male sperm maturation, such as protamine or transition protein, in the somatic donor cell before nuclear transfer.

### FIELD OF THE INVENTION

The invention relates to cloning of non-human animal individuals by Somatic Cells Nuclear Transfer (SCNT). In particular the present invention refers to a method for reconstructing a non-human animal embryo and to a method for obtaining a non-human animal.

### BACKGROUND OF THE INVENTION

SCNT allows the asexual reproduction of individual by transplanting a somatic cell into an enucleated oocyte. SCNT has a tremendous potential as a reproductive technology for both domestic and wild animals. High productive or rare genotypes can be expanded through cloning (Wells D., 2003; Loi P., et al, 2001; Holt W., 2004); non-living animals can be “resuscitated” by nuclear transfer of non viable cells (Loi et al., 2001-2008; Wakayama et al., 2007), and finally, transgenic non-human animals could be produced at higher efficiency by nuclear transfer of genetically modified cells (Robl et al., 2007; Niemann and Kues 2007; Prather et al., 2003).

However, SCNT efficiency is currently very low, and even though there are differences between species, only 1-5% of offspring is usually obtained. The low efficiency of SCNT is a consequence of incomplete nuclear reprogramming of the somatic cell nucleus transplanted into the enucleated oocyte. Nuclear reprogramming refers to the elimination of chromatin modifications associated with cell differentiation by the enucleated oocyte.

Despite many years passed by since the production of the first clone (Wilmut et al., 1997), very little progress has been achieved in non-human animal cloning.

The reasoning behind SCNT failures is that whereas, oocyte reprogramming machinery perfectly fits with spermatozoa nuclei, they lack proper molecular tools to functionally interact with the complex chromatin organization of a somatic cell nucleus.

To improve SCNT, a radical modification of chromatin organization of a somatic cell before nuclear transfer should be induced, as suggested in prior publications (Loi & Cappai, 2000; Loi et al., 2002).

The International Application W000/74477 refers to a process for reconstructing an animal embryo wherein the chromatin in the nucleus of a donor cell is subjected to denaturating conditions before transferring into the recipient cell. The strategies proposed in WO00/74477 for modifying the somatic chromatin is unspecific, and relies on the bulk effects of a thermal treatment to make it more easily reprogrammable by oocyte.

Therefore, there is still the need to provide methods for specifically remodeling the chromatin organization of somatic cells, in order to improve SCNT.

### SUMMARY OF INVENTION

In the present invention, authors have found, for the very first time, that the transient expression of human protamine 1 (NCBI Reference Sequence: NM_002761.2 (SEQ ID NO:1) ; protein, GenBank: AAA63249.1 (SEQ ID NO:2), Rousseaux, et al., 2008; 2011) induces somatic cell nuclei to acquire a structural organization similar to that found in spermatozoa, the cell created for fertilization. The same effect might be elicited simply by inducing an uptake of short synthetic peptides corresponding to the functional part of protamine. Hence, by the process of the invention somatic cell nuclei acquire a spermatozoa like chromatin organization.

“Protaminized” nuclei are then injected into enucleated oocytes and physiologically remodeled by the oocyte's cytoplasm leading to a nuclear reprogramming to the blastocyst stage in high proportion.

Therefore the present invention induces an improvement in nuclear reprogramming in somatic cells nuclear transfer (SCNT), and consequently an improvement in its efficiency.

In the present invention, the modification of somatic chromatin is specific and targeted, for the induced protamine-dependent conformation, like spermatid/spermatozoa in nuclei of somatic cells (never achieved so far), mimics the physiological condition and matches perfectly the reprogramming machinery of the oocyte, resulting in an improved nuclear reprogramming.

Authors demonstrated that protaminized somatic cells (fibroblasts as exemplified in the present invention, but might be any kind of non-human animal cell) display in a repeatable manner (34 replicates) a phenotype overlapping spermatocytes in high proportion (50%) of the transfected cells, within 48 hours post transfection.

The protaminization of somatic cells might be extended to mammalian or not mammalian non-human animals, making the process reliable, with enormous advantages in fields like: non-human animal production and breeding, non-human transgenic animals, endangered non-human animals, and lastly in nuclear reprogramming for regenerative medicine.

Protaminized nuclei are physiologically remodeled by the oocyte, and successfully reprogrammed to the blastocyst stage.

It is therefore an object of the invention a method for reconstructing a non-human animal embryo, comprising the steps of:
a) Transiently expressing into a non-human donor cultivated cell at least a protein involved in male sperm maturation, or a synthetic or recombinant portion thereof able to confer to the chromatin of said cell a spermatozoa like chromatin conformation, or
a′) Inducing the uptake from the cultivating medium of a non-human donor cultivated cell of at least a protein involved in male sperm maturation or a synthetic or recombinant portion thereof able to confer to the chromatin of said cell a spermatozoa like chromatin conformation, and
b) Identifying the nuclei wherein the chromatin has a spermatozoa like chromatin conformation, and
c) Transplanting the identified nuclei into a non-human animal enucleated oocyte to get reconstructed embryo and optionally
d) Culturing said reconstructed embryo in vitro or in vivo.

The uptake of step a′) of the above method is preferably induced by transfection.

The identification of the nuclei of step b) of the above method is preferably carried out by means of fluorescent tag linked protein or by fluorescence-activated cell sorting (FACS) or selecting the nuclei by morphological appearance (elongated structure).

Preferably, the above method according to the invention further comprises the steps of:

e) Transferring said reconstructed embryo into a suitable non-human foster mother/shell.

Said protein involved in male sperm maturation preferably belongs to the following group: very basic DNA-interacting non-histones proteins/peptides, transition proteins (TPS).

Preferably, said very basic DNA-interacting non-histones protein is protamine.

Preferably, said very basic DNA-interacting non-histones protein is protamine 1 or protamine 2.

Preferably the protamine 1 (also defined as Prm1) is human and has essentially the aa sequence present in NCBI database with the GenBank Accession No. AAA63249.1:

| (SEQ ID NO: 2) | |
|---|---|
| 1 | maryrccrsq srsryyrqrq rsrrrrrrsc qtrrramrcc rpryrprcrr h, |

or has an amino acid sequence with a % identity not less than 70% of said aa protamine 1 sequence, or its mRNA is characterized by a cDNA sequence present in NCBI database with the Accession No. NM_002761.2:

| (SEQ ID NO: 1) | |
|---|---|
| 1 | gactcacagc ccacagagtt ccacctgctc acaggttggc tggctcagcc aaggtggtgc |
| 61 | cctgctctga gcattcaggc caagcccatc ctgcaccatg gccaggtaca gatgctgtcg |
| 121 | cagccagagc cggagcagat attaccgcca gagacaaaga agtcgcagac gaaggaggcg |
| 181 | gagctgccag acacggagga gagccatgag gtgctgccgc cccaggtaca gaccgcgatg |
| 241 | tagaagacac taattgcaca aaatagcaca tccaccaaac tcctgcctga gaatgttacc |
| 301 | agacttcaag atcctcttgc cacatcttga aaatgccacc atccaataaa aatcaggagc |
| 361 | ctgctaagga acaatgccgc ctgtcaataa atgttgaaaa gtcatcccaa aaaaaaaaaa |
| 421 | aaaaaa |

or its mRNA is characterized by a cDNA sequence with a % identity not less than 70% of said cDNA protamine 1 sequence.

In another preferred embodiment, protamine 2 is human and has essentially the aa sequence present in NCBI database with the GenBank Accession No.: CAA87066.1:

| (Gene ID: 5620) | |
|---|---|
| (SEQ ID NO: 4) | |
| 1 | mvryrvrsls ershevyrqq lhgqeqghhg qeeqglspeh vevyerthgq shyrrrhcsr |
| 61 | rrlhrihrrq hrscrrrkrr scrhrrrhrr gcrtrkrtcr rh |

or has an amino acid sequence with a % identity not less than 70% of said aa protamine 2 sequence, or its mRNA is characterized by a cDNA sequence present in NCBI database with the Accession No. NM_002762.3:

| (SEQ ID NO: 3) | |
|---|---|
| 1 | agaccagacc aacagtaaca ccaagggcag gtgggcaggc ctccgccctc ctcccctact |
| 61 | ccagggccca ctgcagcctc agcccaggag ccaccagatc tcccaacacc atggtccgat |
| 121 | accgcgtgag gagcctgagc gaacgctcgc acgaggtgta caggcagcag ttgcatgggc |
| 181 | aagagcaagg acaccacggc caagaggagc aagggctgag cccggagcac gtcgaggtct |
| 241 | acgagaggac ccatggccag tctcactata ggcgcagaca ctgctctcga aggaggctgc |
| 301 | accggatcca caggcggcag catcgctcct gcagaaggcg caaaagacgc tcctgcaggc |
| 361 | accggaggag gcatcgcaga ggctgcagaa ccaggaagag aacatgcaga aggcactaag |
| 421 | cttcctgggc ccctcacccc cagctggaaa ttaagaaaaa gtcgcccgaa acaccaagtg |
| 481 | aggccatagc aattccccta catcaaatgc tcaagccccc agctggaagt taagagaaag |
| 541 | tcacctgccc aagaaacacc gagtgaggcc atagcaactc ccctacatca aatgctcaag |
| 601 | ccctgagttg ccgccgagaa gcccacaaga tctgagtgaa atgagcaaaa gtcacctgcc |
| 661 | caataaagct tgacaagaca ctc |

or its mRNA is characterized by a cDNA sequence with a % identity not less than 70% of said cDNA protamine 2 sequence.

Preferably, said transition protein is transition protein 1 (TPS) and transition protein 2 (TPS).

More preferably, transition protein 1 (TPS) is human and has essentially the aa sequence present in NCBI database with the Accession No. NP_003275.1:

| ORIGIN | |
|---|---|
| (SEQ ID NO: 6) | |
| 1 | mstsrklksh gmrrsksrsp hkgvkrggsk rkyrkgnlks rkrgddanrn yrslh, |

//

or has an amino acid sequence with a % identity not less than 70% of said aa transition protein 1 sequence,

or its mRNA is characterized by a cDNA sequence present in NCBI database with the Accession No. NM_003284.3:

| (SEQ ID NO: 5) | |
|---|---|
| 1 | gcccctcatt ttggcagaac ttaccatgtc gaccagccgc aaattaaaga gtcatggcat |
| 61 | gaggaggagc aagagccgat ctcctcacaa gggagtcaag agaggtggca gcaaaagaaa |
| 121 | ataccgtaag ggcaacctga aaagtaggaa acggggcgat gacgccaatc gcaattaccg |
| 181 | ctcccacttg tgagccccca gcgggctctg ccctggtgcg cttcacacag caccaagcag |
| 241 | caacaagaac agcagaaggg gaactgccaa ggagacctga tgttagatca aagccagaga |
| 301 | ggagcctatg gaatgtggat caaatgccag ttgtgacgaa atgaggaatg tatatgttgg |
| 361 | ctgtttttcc ccaacatctc aataaaactt tgaaagcaga aaaaaaaaaa aaaaa |

or its mRNA is characterized by a cDNA sequence with a % identity not less than 70% of said cDNA transition protein 1 sequence.

More preferably, transition protein 2 (TPS) is human and has essentially the aa sequence present in NCBI database with the Accession No. NP_005416.1:

| (SEQ ID NO: 8) | |
|---|---|
| 1 | mdtqthslpi thtqlhsrsq pqsrtctrhc qtfsqscrqs hrgsrsqsss qspashrnpt |
| 61 | gahsssghqs qspntspppk rhkktmnshh spmrptilhc rcpknrknle gklkkkkmak |
| 121 | riqqvyktkt rssgwksn, |

or has an amino acid sequence with a % identity not less than 70% of said aa transition protein 2 sequence,

or its mRNA is characterized by a cDNA sequence present in NCBI database with the Accession No.NM_005425.4:

| (SEQ ID NO: 7) | |
|---|---|
| 1 | aggatgagga ggaggccctg cccctccaaa cgtggcctcc tatggacacc cagactcaca |
| 61 | gccttcctat cacccacact cagctccata gcaactctca gccccaaagc cgcacctgca |
| 121 | cccgccattg ccaaaccttc agccagagtt gcagacagag ccatcgtggc agccggagcc |
| 181 | agagctccag ccagagcccg gccagccacc gcaacccaac tggagcccac agctcatccg |
| 241 | gccaccagag ccagagtccc aacactagtc caccaccaaa gcgccacaaa aagactatga |
| 301 | actcccacca ctctcccatg cggcccacca tcctgcactg ccgctgcccc aagaacagaa |
| 361 | agaacttgga aggcaagctg aaaaagaaaa aaatggccaa gaggatccag caggtgtaca |
| 421 | aaaccaagac gcggagctca ggatggaaat ccaactaatg agaccgcact ccttggcttg |
| 481 | ttcctgcgtg tttcacccaa aggagaaaat gctaggatga agtcaatctt cttgcaggaa |
| 541 | catgttacta tggtgatttc tacgcaacac taattaaagc ttgtacctgg aagactaaa, |

or its mRNA is characterized by a cDNA sequence with a % identity not less than 70% of said cDNA transition protein 2 sequence.

In the instant invention, for % of identity (or % of similarity) it is intended the quantification of the % of elements equal (or similar) in a sequence of a biomolecule (Larkin et at., 2007).

In the method according to the invention the non-human donor cultivated cell is preferably a somatic cell and/or fetal cell. Said somatic cell is a preferably a fibroblast. Other cells might be equally suitable.

Preferably, said non-human donor cultivated cell has been previously genetically modified.

Said genetic modification preferably includes the insertion of at least one heterologous DNA sequence and/or the deletion of at least one homologous gene and/or the modification of at least one homologous gene.

In a preferred embodiment, said non-human donor cultivated cell and the non-human oocyte belong to the same species.

In an alternative embodiment, the non-human donor cultivated cell and the non-human oocyte may belong to different species.

Said species preferably belongs to the group of domestic animals, preferably farm, laboratory, and companion animals, and non mammalian species, preferably avian, amphibian, fish, reptiles.

Another object of the invention is a method for generating a non-human animal, comprising the steps of:
a) Transferring a reconstructed non-human animal embryo as above defined into a suitable non-human recipient animal or intermediate host or eggshell;
b) Causing said non-human animal embryo to develop to term;
d) Further breeding the resulting non-human animal.

The transfer of said reconstructed embryo into a suitable non-human recipient animal or intermediate host, may be an interspecific embryo transfer, i.e. the transfer of an embryo between a different species which is still able to carry the pregnancy to term.

By referring to eggshell it is referred to the application of this procedures to species with external reproduction, like avian/amphibians/reptiles eggshell.

A further object of the invention is a non-human animal obtainable by the method for generating a non-human animal as above defined.

Another object of the invention is a non-human animal reconstructed embryo obtainable by the method for reconstructing a non-human animal embryo as above defined.

In the method of the invention, growing of reconstructed non-human embryos may be carried out to a stage compatible for their transfer into a suitable foster mother/shell.

In the context of the present invention, said synthetic or recombinant portion of the protein involved in male sperm maturation, e.g. of protamine 1, is e.g. from 7 to 25 aa long.

In the definition of “proteins involved in male sperm maturation”, of “protamine 1”, of “protamine 2”, of “transition protein 1” or: of “transition protein 2” are comprised their allelic or orthologous variants, fragments, mutants, orthologues, derivatives or functional analogues. In the present invention, variants, fragments, mutants, orthologues, derivatives or functional analogues possess the same ability to confer to the chromatin of the donor cell a spermatozoa like chromatin conformation.

Preferably the variant or orthologs, derivatives and fragments thereof has at least one residue replaced by a different amino acid residue.

The variants of the present invention, obtained by technologies known in the art, are mutant proteins, which differ from the amino acid sequence of the wild type protein by the mutation of one or more single amino acid. In a very preferred embodiment of the present invention, only one amino acid replacement occurs on the sequence of the native protein. It is, however, encompassed by the subject of the present invention that the native protein can be further optimized by replacement of a plurality, e.g two or more, of amino acid replacements. The variants can therefore differ from the wild type protein sequence by amino acid replacements on 1-10, preferably 1, 2, 3, 4, 5 and 6 different amino acid target positions.

Moreover, the mutants or variants of the invention exhibit the same activity e.g. of protamine 1. The term “mutation” or “variant” as used in the context of the present invention can be understood as substitution, deletion and/or addition of single amino acid in the target sequence. Preferably, the mutation of the target sequence in the present invention is a substitution. The substitution can occur with different genetically encoded amino acid or by non-genetically encoded amino acids. Examples for non-genetically encoded amino acids are homocystein, hydroxyproline, omithin, hydroxylysine, citrulline, carnitine, etc.

As used herein, the term “orthologues” refers to proteins in different species than e.g. the proteins in Homo sapiens that evolved from a common ancestral gene by speciation. As an example of such orthologs, one can cite the proteins corresponding to protamine 1 in Mus musculus, Rattus norvegicus, Pan troglodytes, Bos Taurus, Gallus gallus, Xenopus laevis, Tetraodon nigroviridis, and Danio rerio.

As used herein, the term “derivatives” refers to polypeptides having a percentage of identity of at least 75% with the protein, e.g. with protamine 1, or orthologue thereof, preferably of at least 85%, as an example of at least 90%, and more preferably of at least 95%.

In the present invention, the transient expression of the protein involved in male sperm maturation or of the synthetic or recombinant portion thereof able to confer to the chromatin of said cell a spermatozoa like chromatin conformation, may be obtained by transfection, e.g. by Lipofectamine, with an expression vector, e.g. a plasmid, comprising the protein or portion thereof coding sequence. Said coding sequence may be under the control of a promoter able to efficiently express said coding sequence. Preferably the coding sequence is the sequence consisting essentially of the sequence of SEQ ID No. 1, 3, 5 or 7.

Transfection can be also carried out using calcium phosphate, by electroporation, by cell squeezing, by liposomes, electroporation, gene gun and other current methods of vectors delivery.

In the present invention, the uptake of protein involved in male sperm maturation or of the synthetic or recombinant portion thereof able to confer to the chromatin of said cell a spermatozoa like chromatin conformation may be obtained by electroporation, or other common cell poration techniques (streptolysin 0, Protein uptake kit, etc).

In the context of the present invention it is referred to “reconstructed oocyte” or “reconstructed embryo” indifferently.

The nucleus of the donor cell may be genetically modified prior to the transfer in the recipient cell, in order to obtain non-human transgenic animals. The term “transgenic” refers to a non-human animal containing at least one gene from a different species in their somatic and germ line, and to a non-human animal whose germ line is subjected to technical intervention by recombinant DNA technology, as well.

The present invention will be illustrated by means of non-limiting examples referring to the following figures:

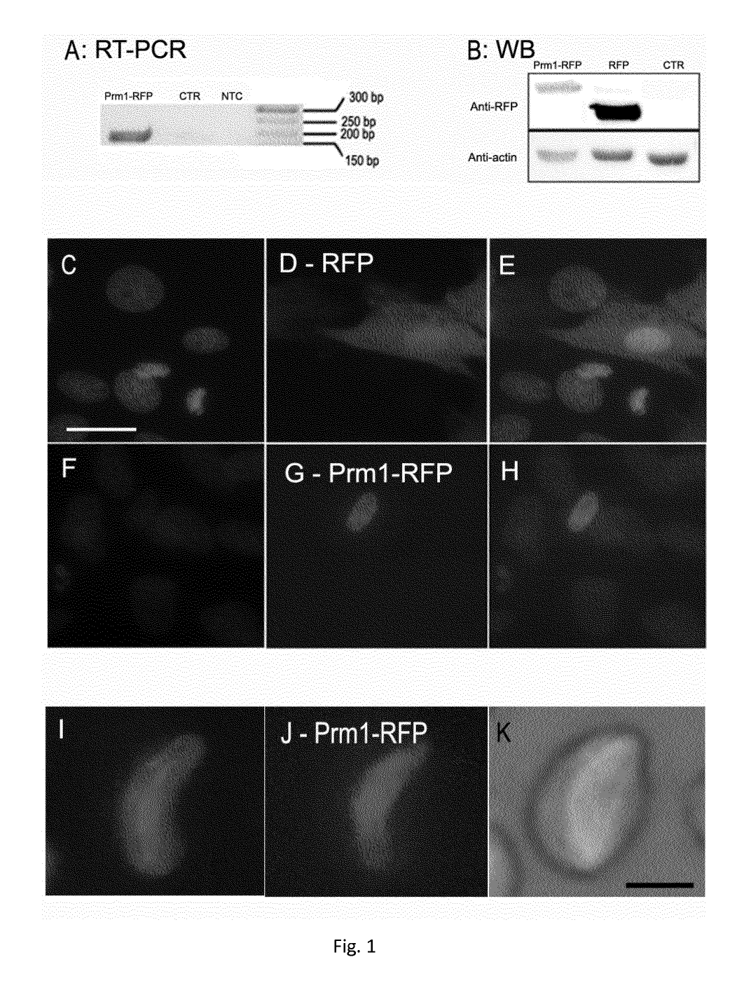

FIG. 1. Expression of protamine 1 gene in adult fibroblasts A—Protamine mRNA expression in sheep adult fibroblasts (RT-PCR). Prm1-RFP: fibroblasts transfected with protamine tagged with RFP. CTR: fibroblasts not transfected. NTC: no template control; B—Protamine protein expression in adult fibroblasts (Western blot, WB). Prm1-RFP: fibroblasts transfected with pPrm1-RFP. pTag-RFP: fibroblasts transfected with empty plasmid-RFP. CTR: fibroblasts not transfected. Immunoblotting with anti-tRFP, anti-actin were used as loading control; Tracking the RFP tag in cells transfected with pTag-RFP (C, D, E) and pPrm1-RFP (F, G, H); Nuclei (Hoechst stained), (C, F), RFP (D) or Prm1-RFP localizations (G), merge (E, H). Scale bar=10 μm; Elongated nucleus in pPrm1-RFP transfected cell (I, J, K). Scale bar=10 μm.

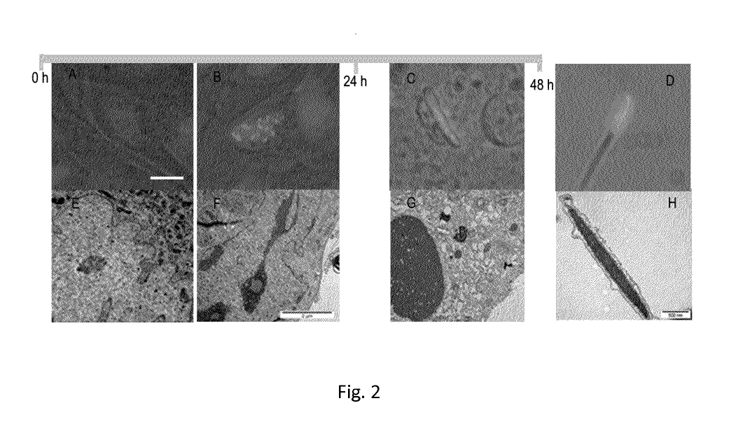

FIG. 2. Timing of incorporation of protamine in somatic nuclei.—Schematic representation of post transfection incorporation of protamine in somatic nuclei. pPrm1-RFP—protamine plasmide tagged with RFP; A—Nucleus of fibroblasts before the transcription of protamine; B—Incorporation of protamine in nucleus 16-20 h post transfection visible as spots; C—Complete incorporation of protamine in nucleus 40-48 h post transfection, complete overlapping nucleus/protamine; E, F, G—Transmission electron microscope (TEM) analysis of adult sheep fibroblasts transfected with pPrm1-RFP; F—Nucleus of fibroblasts before the transcription of protamine., G—Nucleus of fibroblasts after 16-20 h post transfection. Visible partial compaction of chromatin; H—Nucleus of fibroblasts after 40-48 h post transfection. Evident the complete chromatin compaction. Bars=8 μm (A), 2 μm (F); D, H—Nucleus of spermatozoa stained with Hoechst (E) and analyzed by TEM (I). Scale bar: 500 nm

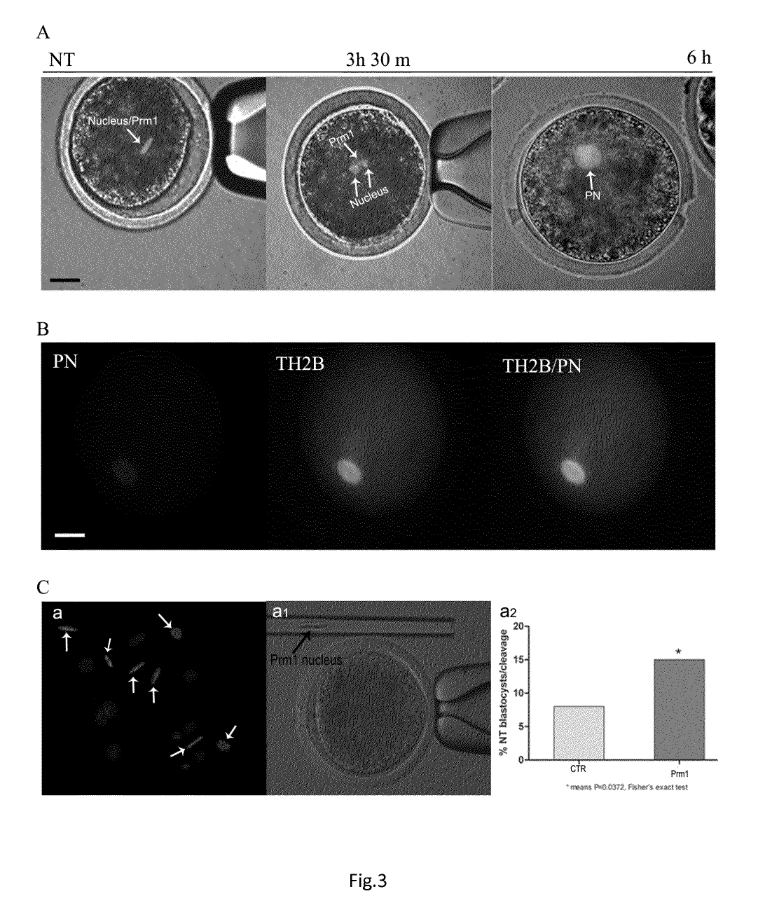

FIG. 3. Nuclear remodelling of protaminized somatic fibroblasts. A-Displacement protamine during pronucleus formation after Nuclear Transfer (NT); scale bar=20 μm. Nucleus/Prm1 (overlapping), 3 h 30 m (displacement Prm1 from nucleus), 6 h (protamine disappears and formation Pronucleus, PN); B- Incorporation TH2B in Pronucleus (PN) after NT of protaminized nuclei. Pronucleus (PN), TH2B , Merge (TH2B/PN). Scale bar=20 μm; C—Somatic cell nuclear transfer (SCNT) of fibroblast transfected with pPrm1-RFP; a—Picture represents protamine (arrows) positive fibroblasts used as donors for SCNT; a1—protaminized nucleus in injected capillar before nuclear transfer into enucleated MII sheep oocytes, a2—Percentage of blastocysts produced by SCNT using control (CTR) and protaminized (Prm) sheep fibroblast as donor.

### EXAMPLE

### Methods

### Plasmids Construction

Sperm protamin 1 (Prm1) cDNA (accession N°: NM_002761.2 [SEQ ID NO:1]) was amplified from a human testis cDNA library with appropriate primes and cloned into a pTagRFP vector (Evrogen, Milan, Italy). The identity of the cloned cDNA and its in frame cloning C-terminal to RFP was verified by sequencing.

### Source of Cells and Transient Transfection.

Sheep Adult Fibroblasts (SAF) were derived from ear biopsy of three female Sarda breed sheep (2 years old). Primary cultures were establishment from the biopsy and fibroblasts were used for transfection experiments between second and eighth passage. SAF were maintained during the culture in Dulbecco's modified eagle medium (DMEM) (Gibco, Life Technology, Milan, Italy) containing 10% FBS (Fetal Bovine Serum), 2 mM Glutamine, 3.7 g/l NaHCO3 and 0.5% Gentamicin. SAF at 80% confluence were transfected with 3 μg of pPrm1-RFP and pRFP (transfection CTR) (pTagRFP vector, FP141, Evrogen, Milan, Italy) by Lipofectamine 2000 (Invitrogen, Oslo, Norway), according to the manufacturer's instructions. The post-transfection medium was changed for DMEM containing 5 nM Trichostatin A (TSA) 4 h post transfection, and SAF were cultured for additional 16, 20, 40, 48 h.

Cells were cultured for 48 hours and checked for phenotypic changes following protamine expression.

The expression of protamine was additionally demonstrated by RT-PCR and western blotting analysis of transfected and control fibroblasts, as described below.

### RNA Isolation and Reverse Transcription (RT-PCR)

Poly (A) +RNA was isolated from cells using Dynabeads mRNA DIRECT kit (Invitrogen Dynal AS, Oslo, Norway) according to the manufacturer's instructions. RT was performed using 80% of the eluted Poly (A) +RNA in a total volume of 20 μl using the QuantiTect Reverse Transcription Kit (Qiagen, Milan, Italy). cDNAs were diluted 1:3 in H₂O. The PCR reactions were performed using the PCR Master Mix (Promega, Milano, Italy). PCR conditions: 95° 5′ (95° C., 30″, 58″ 30° C., 72° C. 30″)×35 cycles, 72° C. 10′. Primers were: FW; atggccagataccgatgct (SEQ ID NO: 9), RV; cagcatcttcgcctcctc (SEQ ID NO: 10); amplicon: 160 bp.

### Western Blotting

Cell protein extracts were denatured by heating at 95° C. for 5 min in 1% (v:v) sodium dodecyl sulphate (SDS), 1% (v:v) β-mercaptoethanol, 20% (v:v) glycerol in 50 mM Tris—HCl at pH 6.8. Samples were subjected to electrophoresis in 10% SDS polyacrylamide gels. After electrophoresis proteins were transferred to nitrocellulose membranes. Membranes were blocked in TBS-T (0.2% (v:v) Tween-20 in 20 mM Tris, 137 mM NaCl at pH 7.6) with 5% (w:v) skimmed milk, and then washed three times in TBS-T at room temperature. Membranes were incubated overnight (o/n) at 4° C. with the primary antibody anti-tRFP (1:300, Evrogen, Milan, Italy) and anti-β-Actin as the loading control (1:1000; sc-1615, Santa Cruz Biotechnology, Santa Cruz, USA) diluted in blocking solution. After three washes with TBS-T, membranes were incubated for 1 h at room temperature with the secondary antibody (anti-goat or anti-rabbit IgG HRP-labelled, Santa Cruz Biotechnology, Santa Cruz, USA) diluted 1:1000 in blocking solution. After three washes in TBS-T, the final detection was performed by enhanced chemiluminescence using the ECL Plus Western Blotting Detection System (Amersham-Pharmacia, Piscataway, USA). Image acquisition was carried out using the ChemiDoc System (Bio-Rad, Milan, Italy). Non transfected cells and transfected with only RFP tag (20 μg/lane) were used as positive controls.

### TEM Analysis

Transfected cells were fixed and processed for TEM (Transmission Electron Microscopy) as described in the following. Cells were washed twice with PBS and fixed in glutaraldehyde (2.5% in 0.1 M cacodylate buffer, pH 7.4) for 24 h. After washing in ddH2O, cells were post-fixed in 2% OsO4 in ddH2O for 4 h and washed three times in ddH2O. Next, cells were dehydrated through a graded series of ethanol solutions (30%-10 min, 50%-15 min, 70%-24 h, 80%-10 min, 96%-10 min, 100%-10 min, acetone—twice for 15 min) and were infiltrated with graded concentrations of EPON resin in 100% acetone (1:3-20 min, 1:1-24 h, 3:1-2 h), infused twice for 1 h in pure EPON resin and polymerized at 65° C. for 24 h. Next, 60 nm sections were prepared and examined using a LEO 912AB electron microscope. Images were captured using a Slow Scan CCD camera (Proscane) and EsiVision Pro 3.2 software (Soft Imaging Systems GmbH).

### Oocyte Maturation

Sheep oocytes recovered from local ovine slaughterhouse were matured in vitro in bicarbonate-buffered TCM-199 medium (Gibco) (275mOsm) containing 2 mM glutamine, 100 mM cysteamine, 0.3 mM sodium pyruvate, 10% foetal bovine serum (FBS) (Gibco), 5 mg/ml FSH (Ovagen), 5 mg/ml LH, and 1 mg/ml estradiol in a humidified atmosphere of 5% CO2/air at 39° C. for 24 h (Ptak G. et al, 2002).

### Nuclear Transfer

Methods of in vitro embryo production were adapted from those previously described (Ptak et al., 2002). Oocytes were matured in vitro in a humidified atmosphere of 5% CO2/air at 39° C. for 24 h. Oocytes were incubated in Hepes-buffered TCM-199 medium containing 4 mg/ml BSA, 7.5 mg/ml Cytochalasin B and 5 mg/ml Hoechst 33342 in an incubator for 15 minutes. Oocytes manipulation was carried out with a piezo-driven enucleation/injection pipette (PiezoXpert, Eppendorf). Enucleation was carried out in Hepes-buffered TCM-199 medium with 0.4% (w/v) BSA and Cytochalasin B with a Narishighe micromanipulator fitted to a Nikon Eclipse inverted microscope. No DNA vital dyes/UV irradiation was used to locate the chromosomes in the oocytes, but a blind aspiration of the cytoplasm surrounding the first polar body was conducted, and enucleation was confirmed later by Hoechst staining and UV irradiation of the aspired cytoplasmic fragments (Iuso et al., 2014). Enucleate oocytes were allowed to recover from the Cytochalasin B treatment and then directly injected with a nucleus, either from CTR or a Prm1-RFP fibroblasts suspended in PBS with 6% Polyvinylpyrrolidone (Sigma). Reconstructed oocytes were activated in Hepes-buffered TCM-199 medium containing 5 mg/ml Ionomycin for 5 minutes and then incubated in SOF medium plus antibiotics and 0.8% BSA containing 10 mM Dimethylaminopurine and 7.5 mg/ml Cytochalasin B for 3-5 hours and cultured for 10-12 hours in SOF enriched with 1% (v:v) minimum essential medium (MEM) nonessential amino acids (Gibco, Milan, Italy), 2% (v:v) basal medium Eagle (BME) essential amino acids, 1 mM glutamine, and 8 mg/ml BSA covered with mineral oil pre-washed in SOF. Cultures were checked for embryonic development every 24 hours till day 7 post activation.

### TH2B Immunostaining on Pronuclear Stage Embryos

The zona pellucida of embryos at pronuclear stage (10-12 h after oocyte activation) was removed by incubation in 0.5% (w/v) pronase and acid Tyrode's solution for 30s. Embryos were then fixed in 4% PFA for 15 min and subjected to immunofluorescence analysis as described in Torres-Padilla et al. (2006) with the rabbit anti-TH2B antibody (1:700, ab23913 Abcam, UK). Finally, zygotes were mounted with VectaShield mounting medium with 5 μg/mL of DAPI.

### Statistical Analysis

Fisher's exact test were used for comparing frequencies of transfection of somatic cells and development to blastocysts to enucleated oocytes injected with protaminized somatic cells.

Probability values lower than 0.05 were considered significant. Statistical analyses were performed using GraphPad Prism 5.0 software.

### Results

The transfected fibroblasts regularly expressed protamine (50%) in 34 replicates (FIG. 1, A,B). Expression of mRNA and protein Prm1 was confirmed by RT-PCR, western blotting and by tracking the RFP-tag (FIG. 1A, B, G). Prm1-RFP co-localized with nuclei stained by Hoechst (FIG. 1F, G, H) whereas in control, pRFP transfected fibroblasts, the red signal was diffused in cytoplasm and nucleus (FIG. 1C, D, E vs F, G, H). Prm1 translocate into the nuclei immediately after its translation and the end product of Prm1 assembly on somatic DNA is the acquisition of a nuclear morphology overlapping that found in elongating spermatids (FIG. 1I, J, K).

Protaminization started as focal points within the nucleus starting 10 hours post transfection, and induced radical nuclear transformation within 48 hours (FIG. 2), when a phenotype overlapping spermatocytes was acquired (FIG. 2C, D). TEM analysis confirmed the extreme compaction of the nuclear compartment (FIG. 2G).

It is concluded that the transient expression of protamine induces a radical chromatin reorganization of fibroblast nuclei (FIG. 2C, G), similar to that occurring in spermatozoa (FIG. 2D, H).

Hence, the method of the present invention confers the chromatin of a somatic cell the structure of a spermatozoa DNA, thus perfectly compatible with the reprogramming machinery of the oocytes.

The remodeling of protaminized cells following nuclear transfer into enucleated oocytes was then analyzed. The protamine red tag was lost following oocyte activation, and the nuclei expand into a normal pronucleus (size: 16.2±2.3 μm) in 89% of the injected oocytes, FIG. 3A. Meanwhile, protamine progressively disappeared in 77% of oocyte injected (28/36, FIG. 3A) and oocyte specific histones variant TH2B began to be assembled in the pronuclei (FIG. 3B). TH2B has recently described as the unique histone variant that plays a key role for nuclear reprogramming (Shinagawa et al., 2014), as it guides the protamine-histone chromatin transition post fertilization (Montellier et al., 2013).

The enucleated oocytes reconstructed with protaminized somatic nuclei were further cultured in vitro in 7 separated replicates, indicating their full competence to direct early embryonic cleavages. Moreover, the developmental frequencies to the blastocyst stage of Prm1 nuclei/derived embryos were significantly higher than control ones (NT blastocysts/cleavage: Prm1, 30/200; 15%; CTR, 14/176; 7.9%) (P=0.0372; Fisher's exact test, FIG. 3, a2).

The protamine-induced reprogramming method object of the present invention is a major breakthrough for nuclear reprogramming. Moreover, the simplicity and the robustness of the protocol developed by the present inventions render the method easily repeatable in all non-human animals reproducible through Somatic Cell Nuclear Transfer.

### REFERENCES

Wells, D″ Reprod Suppl. Vol. 61: 2003, 131-150.
Loi, P. et al. Nat Biotechnol. 2001, 19: 962-964
Holt et al., Reproduction 2004, 127: 317-324
Loi et al., PLOS ONE, 2008 vol. e297
Wakayama T. J Reprod Dev. 2007 53: 13-2.
Robl, et al., Theriogenology, 2007, 67: 127-133.
Niemann, and Kues: Reprod Fertil Dev, 2007, 19: 762-770.
Prather, et al., ‘Theriogenology. 2003, 59: 115-23.
Wilmut I, et., Nature 1997, 385: 810-813
Loi P, Modlinski J A, Ptak G. In Principles of Cloning: Second Edition. Eds. Cibelli J., Wilmut I., Gurdon J. Elsevier. 2013, Pp 353-368.
Loi P, et al., Biol Reprod. 2002 July; 67(1):126-32
Rousseaux S, et al., Reprod Biomed Online. 2008 April; 16(4):492-503.
Rousseaux S, et al., Syst Biol Reprod Med. 2011 February; 57(1-2):50-3.
Ptak, G et al;. Biol Reprod, 2002. 67, 1719-1725.
Iuso, D. et al., Cell Repr. 2013, 15(6), 490-494.
Larkin M. A., et al., (2007) Bioinformatics 23(21): 2947-2948.
Shinagawa, T. et al. Cell Stem Cell, 2014, 14(2):217-27.
Montellier, E., et al. Genes Dev. 2013. 27(15):1680-92.

1. A method for reconstructing a non-human animal embryo, comprising:
a) transiently expressing into a non-human donor cultivated cell a protein involved in male sperm maturation or a synthetic or recombinant portion thereof able to confer to the chromatin of said cell a spermatozoa like chromatin conformation; or
a′) inducing the uptake from the cultivating medium of a non-human donor cultivated cell of at least a protein involved in male sperm maturation or a synthetic or recombinant portion thereof able to confer to the chromatin of said cell a spermatozoa like chromatin conformation,
b) identifying the nuclei wherein the chromatin has a spermatozoa like chromatin conformation;
c) transplanting the identified nuclei into a non-human animal enucleated oocyte to get reconstructed embryo and optionally; and
d) culturing said reconstructed embryo in vitro or in vivo.

2. The method according to claim 1, further comprising the steps of:
e) transferring said reconstructed embryo into a suitable non-human foster mother/shell.

3. The method according to claim 1, wherein the protein involved in male sperm maturation belongs to the group consisting of: very basic DNA-interacting non-histones proteins/peptides, and transition proteins (TPS).

4. The method according to claim 3, wherein the very basic DNA-interacting non-histones protein is protamine 1 or protamine 2.

5. The method according to claim 1, wherein the non-human donor cultivated cell is a somatic cell and/or a fetal cell.

6. The method according to claim 5, wherein the somatic cell is a fibroblast.

7. The method according to claim 1, wherein said non-human donor cultivated cell has been previously genetically modified.

8. The method according to claim 7, wherein said genetic modification includes the insertion of at least one a heterologous DNA sequence and/or the deletion of a homologous gene and/or the modification of at least one a homologous gene.

9. The method according to claim 1, wherein the non-human donor cultivated cell and the non-human oocyte belong to the same species.

10. The method according to claim 9, wherein said species is selected from the group consisting of domestic animals laboratory, and companion animals, and non mammalian species.

11. A method for generating a non-human animal, comprising:
a) transferring a reconstructed non-human animal embryo into a suitable non-human recipient animal or intermediate host or eggshell;
b) causing said non-human animal embryo to develop to term; and
c) further breeding the resulting non-human animal.

12. A non-human animal obtainable by the method according to claim 11.

13. Non-human animal reconstructed embryo obtainable by the method according to claim 1.

14. The method according to claim 10, wherein said domestic animal is a farm animal.

15. The method according to claim 10, wherein said non-mammalian animals are selected from the group consisting of avian, amphibian, fish and reptiles .
